(19) United States
(12) Patent Application Publication
(10) Pub. No.: US 2012/0203278 A1
(43) Pub. Date: Aug. 9, 2012

(54) Crosslink Devices for a Growing Spinal Column Segment

(75) Inventors: Carlos E. Gil, Sunrise, FL (US); Joshua W. Simpson, Collierville, TN (US); Brian A. Butler, Atoka, TN (US); Richard E. McCarthy, Little Rock, AR (US)

(73) Assignee: WARSAW ORTHOPEDIC, INC., Warsaw, IN (US)

(21) Appl. No.: 13/020,989

(22) Filed: Feb. 4, 2011

(51) Int. Cl. A61B 17/70; A61B 17/88

(52) U.S. Cl. 606/250; 606/279

(57) ABSTRACT

The system includes a pair of elongate connecting elements positionable along two or more vertebrae of a spinal column segment. Also included is a crosslink device which is structured to form a mechanical connection between the two connecting elements and is capable of spanning a range of distances separating the two connecting elements to accommodate medial-lateral separation of the connecting elements as the spinal column segment grows over time.

### BACKGROUND

The present invention relates to a prosthetic device and a manner of using the same, and more particularly, but not exclusively, relates to the interconnection of components to assemble an orthopedic construct for treatment of a spinal deformity.

The use of prosthetic implants to address orthopedic injuries and ailments has become commonplace in spinal surgery. In this arena, it is often desired to decrease the invasiveness of the procedures, improve implant integrity, reduce the potential for revision surgery, and provide more positive patient outcomes. Some of these implants depend on interconnection between various system components. Unfortunately, current interconnection devices can be limiting in certain applications. For example, crosslink devices employed to medially-laterally interconnect a pair of spinal rods restrict medial-lateral growth of the spinal column segment to which the rods and crosslink device is attached. Without revision, the crosslink device may constrain growth. Subsequent surgical procedures may be required to replace or adjust the length of the crosslink. Thus, there is a need for additional contributions in this area of technology

### SUMMARY

According to one aspect, a unique prosthesis is provided to interconnect elongate connecting elements that extend along the spinal column while accommodating medial-lateral growth of the spinal column segment to which the prosthesis is connected. Other aspects include unique methods, systems, devices, instrumentation, and apparatus involving an orthopedic stabilization system that reduces or eliminates constraint of medial-lateral growth of the instrumented spinal column segment.

The system includes a pair of elongate connecting elements positionable along two or more vertebrae of a spinal column segment. Also included is a crosslink device which is structured to form a mechanical connection between the two connecting elements and is capable of spanning a range of distances separating the two connecting elements to accommodate medial-lateral separation of the connecting elements as the spinal column segment grows over time.

The crosslink device includes first and second linking members, each including an end member at a first end thereof that is engaged to respective ones of the elongate connecting elements. Each of the first and second linking members also includes an elongate arm extending from the end member toward the other of the first and second linking members. In one embodiment, the elongate arms are engaged in a sliding telescoping arrangement relative to one another. In yet another embodiment, the elongate arms are received in a sliding interfitted arrangement relative to one another. In a further embodiment, each of the arms includes a bracket extending outwardly therefrom adjacent to the end member thereof. Each of the arms is slidingly received in the bracket of the other arm. In one specific embodiment, at least one of the brackets includes a locking mechanism to lock the first and second members in engagement with one another to prevent medial-lateral sliding movement of the first and second arms when it is desired to constrain medial-lateral growth of the spinal column segment, or when the growth of the spinal column segment has reached maturity.

Related features, aspects, embodiments, objects and advantages of the present invention will be apparent from the following description.

### BRIEF DESCRIPTION OF THE DRAWINGS

FIG. 1 is a posterior view of a spinal stabilization system including a crosslink device engaged to the spinal column of a patient.

FIG. 2 is a perspective view of a portion of the spinal stabilization system of FIG. 1.

FIG. 3 is an elevation view of the crosslink device of the spinal stabilization system of FIG. 1.

FIG. 4 is a perspective view of another embodiment crosslink device.

FIG. 5 is a cross-section along line 5-5 of FIG. 4.

FIG. 6 is a perspective view of another embodiment crosslink device.

FIG. 7 is a perspective view of another embodiment of the crosslink device of FIG. 5.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

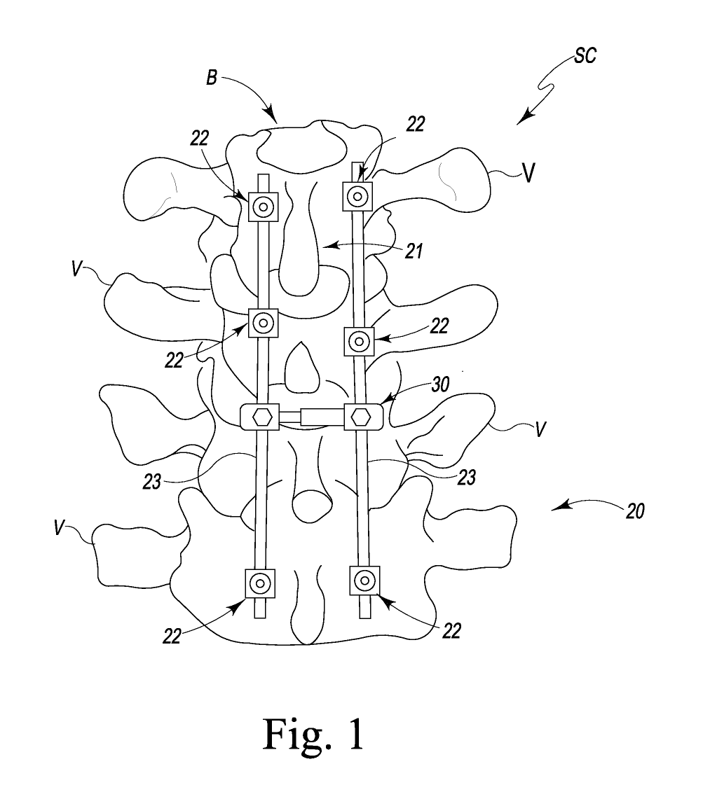

FIG. 1 illustrates a posterior spinal stabilization system 20 implanted at a desired skeletal location of a patient. More specifically, as depicted in FIG. 1, system 20 is affixed to bones B of the spinal column segment SC from a posterior approach. Bones B include at least two vertebrae V of spinal column segment SC, although the posterior spinal stabilization system 20 may be employed in spinal column segments SC including three or more vertebrae V. System 20 includes several bone attachment devices 22 and at least two elongate spinal connecting elements 23 structured to selectively interconnect with bone attachment devices 22. In system 20, bone attachment devices 22 are affixed to various locations of the spinal column 21 and interconnected with connecting elements 23, which are positioned on opposite sides of the medial or sagittal plane of the spinal column to provide bi-lateral stabilization. Bone attachment devices 22 may also be interconnected by one or more crosslink devices 30 to provide a stabilization system for treating spinal disorders. Posterior stabilization system 20 may be used for, but is not limited to, treatment of degenerative spondylolisthesis, fracture, dislocation, scoliosis, kyphosis, spinal tumor, and/or a failed previous fusion associated with spinal column segment SC. Furthermore, spinal column segment SC may comprise any one or combination of the cervical, thoracic, lumbar and sacral regions of the spinal column.

In certain procedures, spinal stabilization system 20 is secured to a spinal column segment SC of a patient to provide stability to a deformed spine while allowing patient growth and without fusion of adjacent vertebrae V. Spinal stabilization system 20 may involve thoracic stabilization in a pediatric orthopedic procedure, although spinal stabilization system 20 is not limited to such procedures. In certain procedures, the patient's spinal column segment SC grows over time in the cephalad-caudal directions and also grows in the medial-lateral directions transversely to the sagittal plane of spinal column segment SC. Crosslink device 30 extends between and is engaged to connecting elements 23 positioned on the opposite sides of the sagittal plane to provide stability to stabilization system 20. Crosslink device 30 lengthens over time in the medial-lateral direction to accommodate growth of spinal column segment SC in the medial-lateral direction while remaining fixed relative to connecting elements 23 in the cephalad-caudal directions to increase the stability of spinal stabilization system 20.

In one specific embodiment, bone attachment devices 22 are bone anchors that are engaged to bony structure of vertebrae V. Bone attachment devices 22 may be, but are not limited to, multi-axial, poly-axial, uni-axial, uni-planar bone screws with a distal bone engaging portion for engaging the bony structure and a proximal receiving portion that is engaged to one of the connecting elements 23. The distal bone engaging portion may be in the form of a bone screw, hook, staple, clamp, tack, wire, adhesive or other suitable bone engaging structure or arrangement. The distal bone engaging portion may be engaged to any suitable bony structure such as a pedicle, lamina, spinous process, transverse process, facet, or anterior portion of a vertebral body. The proximal receiving portion may be a saddle, yoke, post, clamp, eyelet, or other suitable structure that can be engaged to connecting element 23 with or without a separate fastener. In one particular embodiment, the bone engaging portion and receiving portion are movable relative to one another at least initially until engaged to the connecting element 23. In one specific embodiment, the distal bone engaging portion and receiving portion are engaged together with a “ball and joint” or swivel type of coupling that permits relative movement therebetween during at least some stages of assembly.

In addition, connecting element 23 can be in the form of an elongated spinal rod. The spinal rod may be solid or hollow along some or all of its length and/or may be of homogenous or heterogeneous composition. The spinal rod may also be of uniform cross-section along its entire length, or have a variable cross-section along its length. The spinal rod may include one or more interconnected spinal rod portions that lengthen or adjust in length to accommodate growth of spinal column segment SC over time in the cephalad-caudal directions. The spinal rod can be rigid, flexible, or include one or more flexible portions to permit at least limited spinal motion. Other embodiments of connecting element 23 contemplate any suitable spinal stabilization element positionable along the spinal column, including plates, bars, tethers, wires, cables, cords, inflatable devices, expandable devices, and formed in place devices, for example.

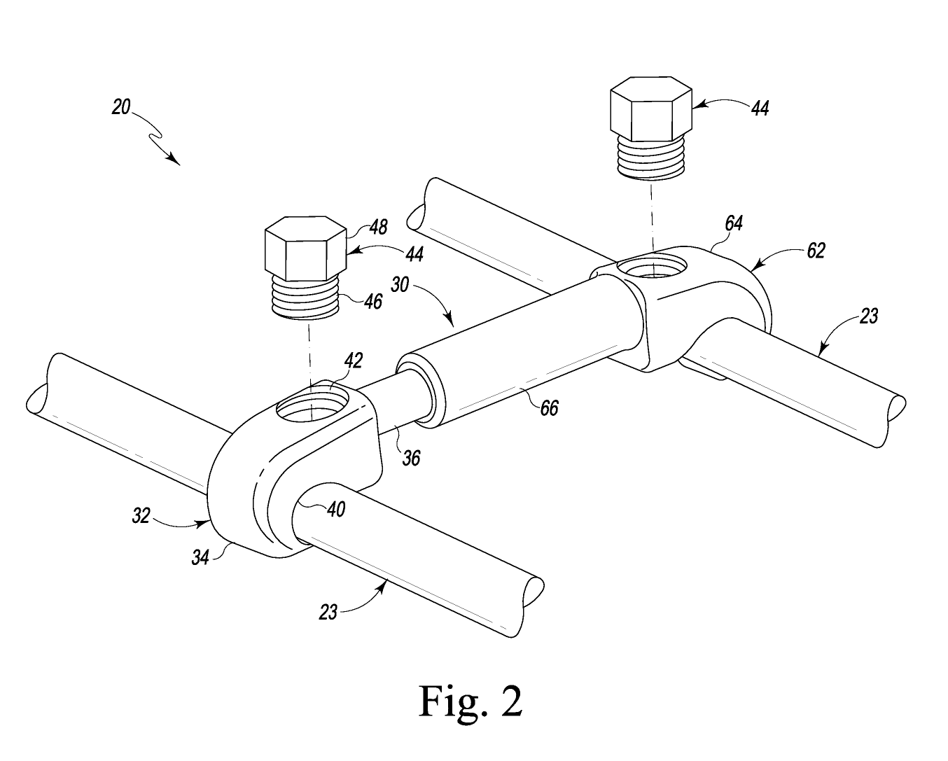

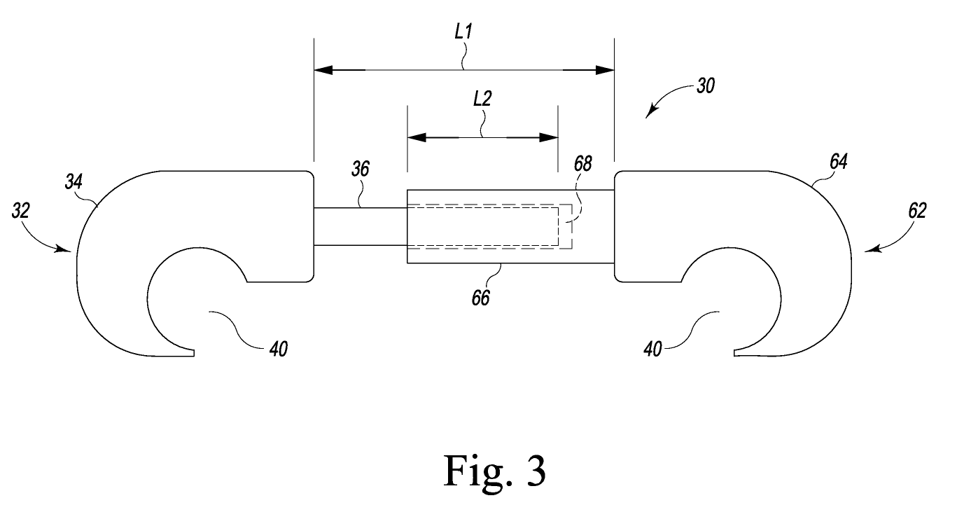

FIGS. 2-3 show one embodiment of crosslink device 30. Crosslink device 30 includes a first linking member 32 and a second linking member 62. First linking member 32 includes a first end member 34 at a first end thereof, and an elongate first arm 36 extending medially-laterally from the end member 34. Second linking member 62 includes a second end member 64 at a first end thereof, and an elongate second arm 66 extending medially-laterally from the end member 64. When end members 34, 64 are engaged to respective ones of the connecting elements 23 engaged to vertebrae V on opposite sides of the sagittal plane, first and second linking members 32, 62 are connected to one another so that first and second arms 36, 66 are positioned in overlapping and sliding relation to one another. This allows end members 34, 64 to move away from one another as connecting elements 23 laterally separate during growth of the spinal column segment SC while first and second linking members 32, 62 remain in engagement with one another to provide stability to stabilization system 20 in the cephalad-caudal and anterior-posterior directions.

End members 34, 64 are shown to be mirror images of one another, although end members 34-64 having different configurations from one another are not precluded. In any event, end member 34 will be described further with it being understood that the description of end member 34 also applies to the illustrated embodiment of end member 64. End member 34 includes a C-shaped body 38 that defines a passage 40 for receiving connecting element 23 in a transverse relation to linking member 32. Connecting element 23 extends cephaladly and caudally from upper and lower ends, respectively, of body 38. Passage 40 also opens along a distal side of body 38 so that connecting element 23 can be bottom-loaded into passage 40 when crosslink device 30 is positioned into the patient and onto connecting elements 23. Body 38 also includes a receptacle 42 opening at a proximal side thereof that intersects passage 40. An engaging member 44 is engageable in receptacle 42 to contact connecting element 23 and secure it against the lateral side of body 38 in passage 40 to fix connecting element 23 in position relative to end member 34.

In the illustrated embodiment, engaging member 44 includes a threaded portion 46 that threadingly engages internal threads along receptacle 42, and is threaded into receptacle 42 to contact connecting element 23 and seat it securely against body 38 of end member 34. Engaging member 44 also includes a tool engaging portion 48 mounted to the proximal end of threaded portion 46 to facilitate installation of engaging member 44. In one specific embodiment, tool engaging portion 48 is removable upon application of a threshold torque to provide a lower profile for crosslink device 30. Other embodiments contemplate other means for securing connecting element 23 in passage 40, including set screws without break-off portions, clamps, and bands, for example. In other embodiments, body 23 provides a snap fit or interference fit with connecting element 23 in passage 40. It is further contemplated that end member 34 can be configured other than with the distally opening passage 40 shown in FIG. 2. For example, passage 40 can open laterally, proximally, obliquely, or medially to receive connecting element 23 therein. In still other embodiments, passage 40 is completely encircled by body 38 so that connecting element 23 is loaded endwise into passage 23.

First linking member 32 includes first elongate arm 36 extending from a medial side of body 38 toward second linking member 62. In addition, second linking member 62 includes second elongate arm 66 extending toward and telescopingly receiving first elongate arm 36. As further shown in FIG. 3, second arm 66 includes a sleeve-shaped body with an internal bore 68 extending axially therein that opens at the second end of second linking member 62. Internal bore 68 is sized to receive an elongated body of first arm 36 in a close fit to restrict cephalad and caudal movement of crosslink device 30 at its center, but allowing sliding movement of arms 36, 66 relative to one another. Therefore, first linking member 32 is not clamped or otherwise fixed in a medial-lateral position relative to second linking member 62 when implanted. In the illustrated embodiment, the cross-section of first arm 36 and second arm 66 defines a circle, and bore 68 has a cross-section that is a circle. Other embodiments contemplate other cross-sectional shapes for one or more of the outer perimeter of arm 36, arm 66 and bore 68, including ovals, non-circular, elliptical, square, rectangular, polygonal, and irregular shapes.

First and second arms 36, 66 separate end members 34, 64 by a length L1 that varies as end members 34, 64 move away from one another as a result of medial-lateral growth of spinal column segment SC. In addition, bore 68 provides an overlap length L2 between arms 36, 66 that is sufficient to accommodate the anticipated amount of medial-lateral growth of the spinal column segment SC without causing linking members 32, 62 to become disengaged from one another. Furthermore, overlap length L2 provides stability in the cephalad-caudal directions by preventing or resisting pivoting movement of linking members 32, 62 relative to one another in a plane that includes or is along the plane that is defined by connecting elements 23. The telescoping and overlapping arrangement also prevents pivoting in the anterior-posterior directions toward and away from spinal column segment SC. In one specific embodiment, arms 36, 66 are configured so that overlap length L2 is at least 50% of length L1 when crosslink device is initially implanted. In a further embodiment, overlap length L2 is at least 50% of length L1 at the greatest anticipated L1 that will result due to medial-lateral growth of spinal column segment SC.

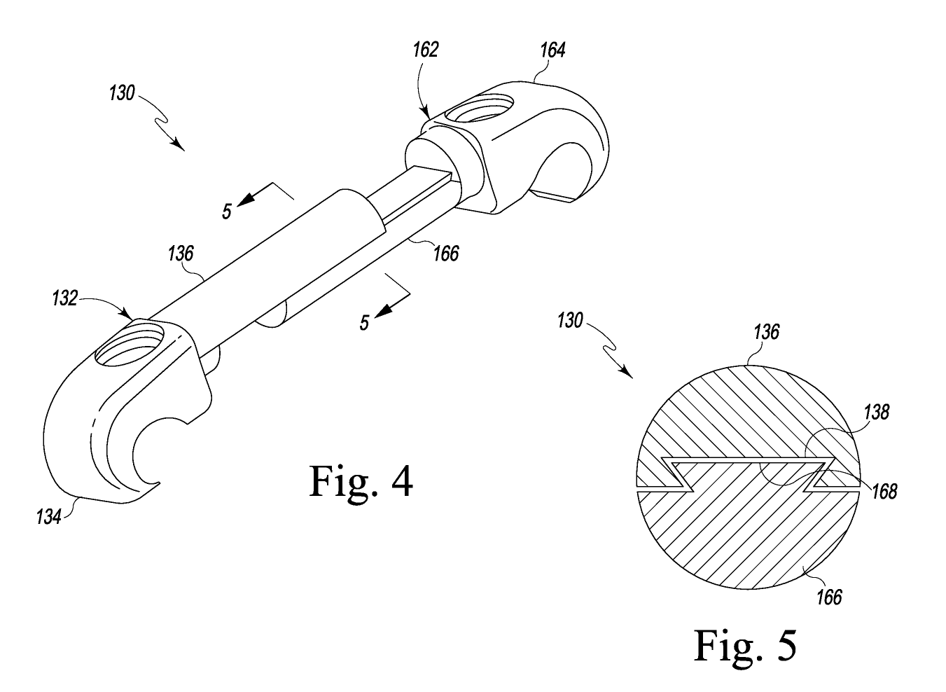

FIGS. 4-5 show another embodiment of crosslink device 30 designated as crosslink device 130. Crosslink device 130 includes first and second linking members 132, 162 having end members 134, 164, respectively. End members 134, 164 can be identical to end members 34, 64 discussed above, or include any suitable configuration for engagement to respective ones of the connecting elements 23. First linking member 132 also includes a first elongate arm 136 extending medially-laterally from end member 134, and second linking member 162 includes a second elongate arm 166 extending medially-laterally from end member 164. First and second elongate arms 136, 166 are engaged to one another in overlapping and sliding relation with an interfitted connection arrangement. As shown in FIG. 5, first arm 136 includes a central groove 138 formed in a side of first elongate arm 136 that faces second elongate arm 166. Second elongate arm 166 includes a dovetail-shaped projection 168 extending from a side of second elongate arm 166. Projection 168 extends into the correspondingly shaped groove 138. The dovetail connection allows movement of linking members 132, 162 away from one another to accommodate medial-lateral growth of the spinal column segment SC. The overlapping, interfitted connection provided by the dovetail arrangement prevents cephalad-caudal pivoting of linking members 132, 162 relative to one another in or along a plane that is defined by connecting elements 23. Furthermore, the dovetail connection restricts or prevents linking members 132, 162 from pivoting relative to one another in the anterior-posterior directions toward or away from spinal column segment SC. In other embodiments, the interfitted groove and projection of arms 136, 166 are arranged in other than a dovetail, such as a T-shaped projection received in a T-shaped groove, an L-shaped arrangement, or a bulbous projection received in a rounded groove, for example.

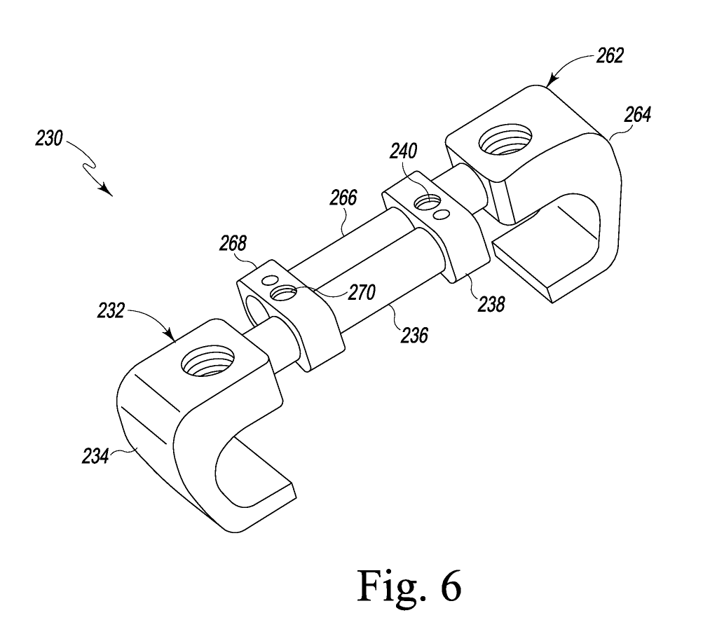

FIG. 6 shows another embodiment of crosslink device 30 designated as crosslink device 230. Crosslink device 230 includes first and second linking members 232, 262 having end members 234, 264, respectively. End members 234, 264 can be identical to end members 34, 64 discussed above, or include any suitable configuration for engagement to respective ones of the connecting elements 23. First linking member 232 also includes a first elongate arm 236 extending medially-laterally from end member 234, and second linking member 262 includes a second elongate arm 266 extending medially-laterally from end member 264. First and second elongate arms 236, 266 extend side-by-side relative to one another and are engaged to one another in an offset and overlapping relation with a bracketed connection. In the illustrated embodiment, arms 236, 266 are positioned side-by-side so that one is located caudally of the other. In other embodiments, arms 236, 266 are positioned side-by-side so that one is located anteriorly of the other, or located anterior and either caudally or cephaladly of the other.

First elongate arm 236 includes a first bracket 238 extending cephaladly from an outer end of first elongate arm 236. Second elongate arm 266 includes a second bracket 268 extending caudally from an outer end of second elongate arm 266. Second elongate arm 266 is slidingly received through a bore of first bracket 238 so that first bracket 238 is located at an outer end of second elongate arm 266 that is adjacent to second end member 264. First elongate arm 236 is slidingly received through a bore of second bracket 268 so that second bracket 268 is located at an outer end of first elongate arm 236 that is adjacent to first end member 234. The spinal column segment SC grows in a medial-lateral direction, elongate arms 236, 266 slide in respective ones of the brackets 268, 238 to allow end members 234, 264 to separate as the connecting elements 23 engaged to end members 234, 264 laterally separate. The cephalad-caudally extending brackets 238, 268 prevent cephalad-caudal pivoting of linking members 232, 234 relative to one another and anterior-posterior pivoting of linking members 232, 234 relative to one another. Brackets 238, 268 can be formed monolithically with the respective elongate arm 236, 266, or formed as separate components that is fastened to the respective elongate arm 236, 266 via fasteners, welded connections, rivets, adhesives, or other suitable fastener or joining technique.

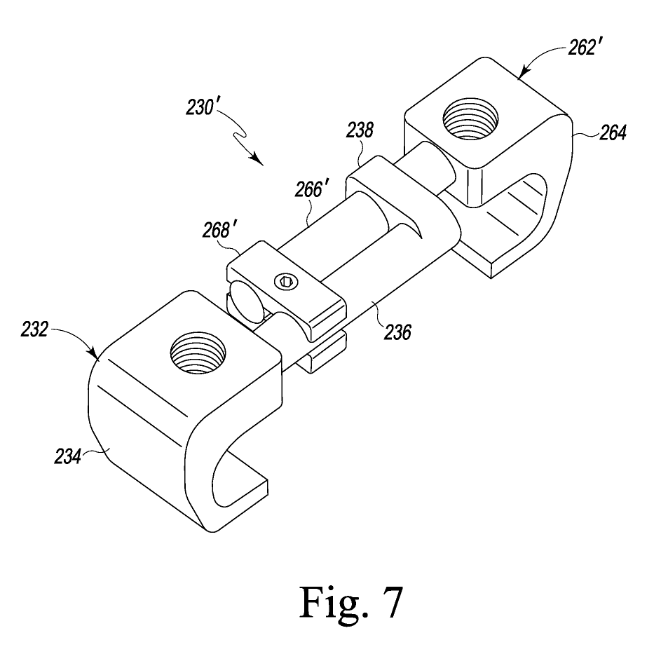

In FIG. 7 there is shown a modified crosslink device 230′ that is similar to crosslink device 230, but in which second bracket 268 of the second linking member is replaced with a locking mechanism 268′. Locking mechanism 268′ allows the second linking member 262′ to be locked into position on first elongate arm 236 to prevent medial-lateral movement of linking members 232, 262′ away from one another. Locking mechanism 268′ can be implanted at least initially in an unlocked condition, and then locked into position in a second procedure to provide additional stability to the stabilization system when medial-lateral growth has reached maturity or no longer needs to be accommodated by the crosslink device. Locking could also be achieved by providing one or both of brackets 238, 268 with a receptacle 240, 270 to receive a set screw or other engaging member to engage the corresponding arm 266, 236 received therein, as shown in FIG. 6.

Materials for the implants disclosed herein can be chosen from any suitable biocompatible material, such as titanium, titanium alloys, cobalt-chromium, cobalt-chromium alloys, stainless steel, PEEK, polymers, or other suitable metal or non-metal material and combinations and composites thereof. Of course, it is understood that the relative size of the components can be modified for the particular vertebra(e) to be instrumented and for the particular location or structure of the vertebrae to which the anchor assembly will be engaged.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A spinal stabilization system, comprising:
a first elongated connecting element and a second elongate connecting element, said first and second elongated connecting elements being positionable along opposite sides of a medial plane of a spinal segment in a cephalad-caudal direction and each is engageable to at least one vertebra of the spinal column segment with a bone anchor;
a crosslink device positionable across the medial plane and engageable to said first and second elongated connecting elements to link said first and second connecting elements to one another, said crosslink device including:
a first linking member having a first end member engageable to said first connecting element and a first elongated arm extending from said first end member toward said second connecting element;
a second linking member having a second end member engageable to said second connecting element and a second elongated arm extending from said second end member toward said first connecting element; and
wherein said first and second elongated arms are engaged in overlapping and sliding relation so that when said first and second end members are fixedly engaged to said first and second connecting elements, said first and second elongated arms slide relative to one another as said first and second elongate members move away from one another in response to medial-lateral growth of the spinal column segment while pivoting of said first and second linking members toward and away from the spinal column segment and in the cephalad-caudal direction is prevented by engagement of said first and second elongated arms with one another.

2. The system of claim 1, wherein when said first and second end members are engaged to said first and second connecting elements, a first length is defined from said first end member to said second end member and overlapping portions of said first and second elongated arms define a second length, said second length always being at least one half of said first length throughout a range of expected medial-lateral growth of the spinal column segment.

3. The system of claim 1, wherein said second elongated arm includes a sleeve-shaped body defining a longitudinal bore opening at an end of said sleeve-shaped body and said first elongated arm includes an elongated body slidingly received in said bore of said sleeve-shaped body of said second elongated arm and overlap of said elongated body in said sleeve-shaped body prevents pivoting of said first and second linking members relative to one another.

4. The system of claim 1, wherein said first elongated arm includes an elongated groove extending along one side thereof and said second elongated arm includes an elongated projection extending from one side thereof that is slidingly received in said groove of said first elongated arm, said groove and projection mating in an elongated interfitted connection to prevent pivoting of said first and second linking members relative to one another.

5. The system of claim 1, wherein:
said first and second elongated arms are positioned in side-by-side relationship to one another;
said first elongated arm includes a first bracket projecting outwardly therefrom toward said second elongated arm, said first bracket defining a bore that slidingly receives said second elongated arm therethrough; and
said second elongated arm includes a second bracket projecting outwardly therefrom toward said first elongated arm, said second bracket defining a bore that slidingly receives said first elongated arm therethrough.

6. The system of claim 5, wherein:
said first elongated arm extends from said first end member to a first end opposite said first end member and said first bracket is located at said first end of said first elongated arm; and
said second elongated arm extends from said second end member to a second end opposite said second end member and said second bracket is located at said second end of said second elongated arm.

7. The system of claim 6, wherein at least one of said first and second brackets includes a receptacle for receiving a fastener to lock said first and second arms in position relative to one another.

8. The system of claim 1, wherein each of said first and second end members defines a passage for receiving a respective one of said first and second connecting elements therethrough.

9. The system of claim 8, wherein each of said first and second end members defines a receptacle that opens into said passage thereof and further comprising first and second engaging members engageable in respective ones of said receptacles of said first and second end members to fixedly engage a corresponding one of said first and second connecting elements in said passage thereof.

10. A spinal stabilization system, comprising:
a first elongated connecting element and a second elongate connecting element, said first and second elongated connecting elements being positionable along opposite sides of a medial plane of a spinal segment in a cephalad-caudal direction and each is engageable to at least one vertebra of the spinal column segment with a bone anchor;
a crosslink device positionable across the medial plane and engageable to said first and second elongated connecting elements to link said first and second connecting elements to one another, said crosslink device including:
a first linking member having a first end member engageable to the first connecting element and a first elongated arm extending from said first end member toward said second connecting element;
a second linking member having a second end member engageable to the second connecting element and a second elongated arm extending from said second end member toward said first connecting element; and
wherein said first and second elongated arms are engaged in overlapping and sliding relation so that when said first and second end members are fixedly engaged to the first and second connecting elements a first length is defined from said first end member to said second end member and overlapping portions of said first and second elongated arms define a second length, said second length always being at least one half of said first length throughout a range of expected medial-lateral growth of the spinal column segment.

11. The system of claim 10, wherein said first and second elongated arms slide relative to one another as said first and second elongate members move away from one another in response to medial-lateral growth of the spinal column segment while pivoting of said first and second linking members toward and away from the spinal column segment and in the cephalad-caudal direction is prevented by engagement of said first and second elongated arms to one another.

12. The system of claim 10, wherein said second elongated arm includes a sleeve-shaped body defining a longitudinal bore opening at an end of said sleeve-shaped body and said first elongated arm includes an elongated body slidingly received in said bore of said sleeve-shaped body of said second elongated arm and overlap of said elongated body in said sleeve-shaped body prevents pivoting of said first and second linking members relative to one another.

13. The system of claim 10, wherein first elongated arm includes an elongated groove extending along one side thereof and said second elongated arm includes an elongated projection extending from one side thereof that is slidingly received in said groove of said first elongated arm, said groove and projection mating in an elongated interfitted connection to preventing pivoting of said first and second linking members relative to one another.

14. The system of claim 10, wherein:
said first and second elongated arms are positioned in side-by-side relationship to one another;
said first elongated arm includes a first bracket projecting outwardly therefrom toward said second elongated arm, said first bracket defining a bore that slidingly receives said second elongated arm therethrough; and
said second elongated arm includes a second bracket projecting outwardly therefrom toward said first elongated arm, said second bracket defining a bore that slidingly receives said first elongated arm therethrough.

15. The system of claim 14, wherein:
said first elongated arm extends from said first end member to a first end opposite said first end member and said first bracket is located at said first end of said first elongated arm; and
said second elongated arm extends from said second end member to a second end opposite said second end member and said second bracket is located at said second end of said second elongated arm.

16. A method for stabilizing a spinal column segment, comprising:
positioning a first elongated connecting element along a first side of the spinal column segment;
engaging the first connecting element to at least one vertebra of the spinal column segment;
positioning a second elongated connecting element along a second side of the spinal column segment opposite the first side;
engaging the first connecting element to at least one vertebra of the spinal column segment;
securing a crosslink device to each of the first and second connecting elements so that the crosslink device extends between the connecting elements, the crosslink device including a first linking member engaged to the first connecting element and a second linking member engaged to the second connecting element, the first and second linking members including first and second elongated arms, respectively, wherein the first and second elongated arms overlap one another and are engaged in sliding relation to one another between the first and second connecting elements; and
sliding the first and second arms relative to one another as the first and second connecting elements separate in response to medial-lateral growth of the spinal column segment while preventing pivoting of the first and second linking members toward and away from the spinal column segment and while preventing pivoting of the first and second linking members in the cephalad-caudal direction.

17. The method of claim 16, wherein the second elongated arm includes a sleeve-shaped body defining a longitudinal bore opening at an end of the sleeve-shaped body and the first elongated arm includes an elongated body slidingly received in the bore of the sleeve-shaped body of the second elongated arm, the overlapping positioning of the elongated body in the sleeve-shaped body preventing pivoting of the first and second linking members relative to one another.

18. The method of claim 16, wherein the first elongated arm includes an elongated groove extending along one side thereof and the second elongated arm includes an elongated projection extending from one side thereof that is slidingly received in the groove of the first elongated arm, the groove and projection mating in an elongated interfitted connection to preventing pivoting of the first and second elongated arms relative to one another.

19. The method of claim 16, wherein:
the first and second elongated arms are positioned in side-by-side relationship to one another;
the first elongated arm includes a first bracket projecting outwardly therefrom toward the second elongated arm, the first bracket defining a bore that slidingly receives the second elongated arm therethrough; and
the second elongated arm includes a second bracket projecting outwardly therefrom toward the first elongated arm, the second bracket defining a bore that slidingly receives the first elongated arm therethrough.

20. The method of claim 19, wherein:
the first elongated arm extends from the first end member to a first end opposite the first end member and the first bracket is located at the first end of the first elongated arm; and
the second elongated arm extends from the second end member to a second end opposite the second end member and the second bracket is located at the second end of the second elongated arm.
